(19) United States
(12) Patent Application Publication
(10) Pub. No.: US 2005/0005320 A1
(43) Pub. Date: Jan. 6, 2005

(54) Recombinant plants mimicking the activity of the hp-1 mutant from tomato

(76) Inventor: Catherine Ann Shipton, Berkshire (GB)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/433,318

(22) PCT Filed: Nov. 30, 2001
(86) PCT No.: PCT/GB01/05313

(30) Foreign Application Priority Data
Dec. 6, 2000 (GB) 0029795.2

(51) Int. Cl. C12N 15/82; C12N 15/87; A01H 5/00; C12N 15/09; C12N 15/63; A01H 5/10

(52) U.S. Cl. 800/278; 800/287; 435/468; 800/295; 800/298; 435/320.1

(57) ABSTRACT

A method for increasing the phytonutrient and/or chlorophyll content and/or yield of a crop, said method comprising transforming a plant cell from which viable plants maybe recovered, with a DNA construct comprising a sequence which encodes phytochrome A or a moiety which interacts with the phytochrome A pathway so as to mimic the activity of the hp-1 mutant of tomato, said sequence being under the control of a tissue specific promoter which is specific for a crop tissue of the plant, and thereafter generating viable plants from said cell. Various means of achieving this are described.

The present invention relates to methods of producing recombinant plants which are high yielding and have elevated carotenoid levels in their crop parts such as the fruits, as compared to the wild-type, as well as to DNA constructs and cells used in their preparation, and to novel plants obtained thereby.

Dietary antioxidants such as vitamin A form an important element in the constitution of a healthy diet. Vitamin A is derived from carotenoids found in many foodstuffs and in particular crops such as fruits like tomatoes and vegetables like carrots. It would be advantageous, particularly in countries where diets are generally lacking in these elements, if varieties of crops could be grown which had elevated carotenoid levels over and above that found in wild-type varieties. Such varieties would constitute so-called “functional foods” or “neutraceuticals”.

In addition, the colour of food affects the aesthetic quality and therefore the desirability of the product. For example, vegetables such as lettuce are more appealing if they are green. The green coloration is caused by chlorophyll in the leaves and consequently, increasing the chlorophyll levels would enhance the appeal of such vegetables.

Lycopene is the red carotenoid found in tomatoes. Extensive epidemiological evidence indicates that lycopene in the diet is associated with a reduced risk of cancers, in particular prostate cancer, and a reduction in the risk of heart disease.

A number of natural high pigment strains of tomato are known. These include hp-1 and hp-2 mutants both of which have high levels of carotenoids in the fruit compared to the wild type.

Furthermore, the incorporation of the hp allele into tomato varieties by breeding is not itself a useful commercial proposition since it is associated with a number of undesirable non-fruit phenotypes such as brittle stems, thick leaves, dwarfism and low fertility. This has prevented its incorporation into breeding programs to date.

The High-Pigment-1—mutant (hp-1) is described by J. L. Peters et al., Plant Physiol. (1998) 117:797-807. It has been found that three light regulated genes, chlorophyll a/b-binding protein (CAB), ribulose-1,5-bisphosphate carboxylase/oxygenase small subunit and chalcone synthase (CHS) are upregulated in this strain. It has been hypothesised that the HP-1 protein may play a general repressive role in phytochrome signal transduction.

Further, the hpl mutation in tomato is known to result in increased plastid DNA in hypocotyls (Yen et al., 1997, Theor. Appl. Genet.). However, it is generally accepted that the ratio between cell volume and total plastid volume per cell is fixed (see Pyke, 1999, Plant Cell 11:549-56).

The tomato HP1 gene has been shown to encode a negative regulator specific for phytochrome A signal transduction (Peters et al., 1992, Photochem. Photobiol.; see Giovannoni et al., 1998, Genetic and Environmental Manipulation of Horticultural Crops).

However the HP-1 gene itself has not been isolated.

EP-A-0354687 describes overexpression of phytochrome in transgenic plants to produce some desirable traits such as increased shade tolerance and dark green colour.

Overexpression of phytochrome, and in particular oat (Avena) phytochrome, in tomato has been described as giving rise to a visual plant phenotype very similar to that of the hp tomato mutants (i.e. dwarfing, dark green foliage and fruits, short hypocotyl and increased anthocyanin levels (M. T. Boylan et al. The Plant Cell, 1, 765-773, (1989)).

The applicants have further found that the carotenoid content of these transgenic tomatoes is significantly higher than wild-type. Furthermore, it has been found that the cell size found in the leaves of these plants is greater. This gives rise to the possibility that these plants may be higher yielding.

According to the present invention there is provided a method for increasing the phytonutrient and/or chlorophyll content and/or yield of a crop, said method comprising transforming a plant cell from which viable plants maybe recovered, with a DNA construct comprising a sequence which encodes phytochrome A or a moiety which interacts with the phytochrome A pathway so as to mimic the activity of the hp-1 mutant of tomato, said sequence being under the control of a tissue specific promoter which is specific for a crop tissue of the plant, and thereafter generating viable plants from said cell.

The term “crop tissue” used herein refers to that part of the plant that is intended for use as a foodstuff or feed, or fibres (such as cotton fibres). The crop may be a horticultural or field crop. Thus in tomatoes, the crop tissue will be the fruit and the tissue specific promoter employed will be a fruit specific promoter. In other crops, this may be the leaf tissue (for example, lettuce, cabbage and spinach), or seed tissue (e.g. in grain such as maize or wheat) or root tissue (in vegetables such as carrots, and potatoes) and the promoter is selected accordingly.

Phytonutrients which may be increased as a result of the use of the method of the invention include carotenoids, anthocyanins, or vitamins, and in particular carotenoids. Increasing the content of these will enhance the nutritional value and may also improve the aesthetic quality of the crop. Increasing chlorophyll content may also fulfil the latter criteria.

Crops modified in accordance with the invention may show increased firmness.

The applicants have found that the hpl gene is the only gene to alter the ratio between cell volume and total plastid volume per cell and thereby produce a fruit containing a higher carotenoid level than wild-type (WT). An increase in the plastid area/cell would be expected to increase the amount of components that accumulate in plastids. These include nutritious compounds such as carotenoids, vitamin E etc. and thus the nutritional value of the plants will improve. In fact, the hp-1 mutant itself shows a two-fold increase in carotenoid level.

Suitably in order to mimic the hp-1 phenotype, either phytochrome A expression is increased, and/or the activity of one or more signalling molecules in the phytochrome A transduction pathway are affected so as to increase the activity of phytochrome A or its signal transduction pathway. In particular, the activity of a signalling molecule which acts as a negative regulator of phytochrome A signal transduction is down-regulated.

Thus according to the present invention there is provided a method for increasing the phytonutrient and/or chlorophyll and/or yield of a crop, said method comprising transforming a plant cell from which viable plants maybe recovered, with a DNA construct comprising either (i) a sequence which encodes phytochrome A, and/or (ii) a sequence which encodes a moiety capable of downregulating a negative regulator of phytochrome A signal transduction, or (iii) a sequence which encodes a moiety capable of upregulating a positive regulator of phytochrome A signal transduction; wherein sequences (i), (ii) and/or (iii) are under the control of a tissue specific promoter which is specific for a crop tissue of the plant, and thereafter generating viable plants from said cell and recovering crop from the plant.

In a further embodiment, the HP1 gene itself is downregulated using conventional methods, for example by complete or partial deletion or mutation including insertional mutation of the HP1 gene, as well as the expression of sense or antisense RNA constructs or oligonucleotides. Identification of the HP1 gene can be carried out using routine methods.

Using the method of the invention, the crop of the transformed plants will mimic the beneficial properties of the hp-1 mutant of tomato, but the problems of the phenotype will be avoided. In particular, by using a crop tissue specific promoter, the hp-1 phenotype is expressed only in the crop parts of the plant such as the fruit, leaves or roots, and the problems associated with the dwarfism and other stem and leaf malformations is avoided. In addition, such plants would be expected to have normal fertility.

Analysis of chloroplast and chromoplast development in the high pigment-1 mutant of tomato has revealed an increase in the plastid compartment size in both leaf mesophyll and fruit pericarp cells. This strongly suggests that the increased pigment levels in the hp-1 mutant (Table 1 below) are, at least in part, a result of increased plastid compartment size per cell, thus providing a larger compartment for carotenoid and chlorophyll synthesis and deposition.

As a result of the increased cell size or plastid number, plants transformed using the method of the invention will be higher yielding than the wild-type varieties. An increase in plastid compartment size, however, is not as extensive as the 1.8-fold increase in carotenoids in hp-1 fruit (Table 1). Consequently, hp-1 contains proportionately more pigment in its larger chromoplasts. In contrast, no plastid compartment volume change has been observed with the hp-2 mutant.

Another contributory factor is the increase in the phytoene synthase activity in ripe fruit plastids. Previous studies have shown that this enzyme, the first in the carotenoid pathway, increases dramatically in breaker fruit, at the onset of lycopene synthesis (Fraser et al., Plant Physiol (1994) 105: 405-413). This has lead to the suggestion that it is a rate-limiting step in carotenogenesis. Thus, the increase shown in hp-1 facilitates flux into all unsaturated carotenoids. Since the profile of carotenoids is the same in hp-1 and wild type (Table 2), a general effect on all carotenoids occurs in this mutant.

Since the relative expression of Psy-1 and Pds does not alter in the fruit of hp-1. compared with the wild type the increase in phytoene synthase enzyme activity is presumably due to an improved utilisation of mRNA in hp-1. This is one of the pleiotropic effects characteristic of enhanced phytochrome responses in plants (Hart, 1988 Light and Plant Growth, Unwin Hyman, London).

The report of a five-fold increase in the ratio of plastome:genomic DNA in hypocotyls of the hp-1 mutant led to the hypothesis that HP-1 regulates plastid DNA copy number in response to light (Yen et al., 1997 supra.). A component of this increased ratio may be manifested via an increased plastid compartment size in hp-1, as observed by the applicants in the present study. Changes in the endoreduplication profile in hypocotyl cells, which occurs in photomorphogenic mutants (Gendreau et al., Plant J. (1998) 13: 221-230) are likely to have a significant effect on any experimentally determined DNA ratio.

It has been found that leaf pigmentation in hp-1 leaves is increased which appears to be due to both increased leaf thickness resulting from greater periclinal expansion of palisade mesophyll cells and increased plastid cover in both palisade and spongy mesophyll cells. Increased leaf thickness would give rise to an increase in specific leaf weight in hp-1 leaves which has been reported previously (Sanders et al., Hort. Sci. (1975) 10: 262-264). The increased palisade cell elongation in hp-1 leaves is a classic photomorphogenic response to light intensity, often manifested in leaves grown in sun or shade (Lichtenthaler (1985) In NR Baker et al., Eds, Control of Leaf Growth, Cambridge University Press, Cambridge University Press pp201-202) or under different light intensities (Pyke and Lopez-Juez, Critical Reviews in Plant Science (1999) 18:527-546).

In addition, sub-palisade mesophyll cells can also show periclinal elongation in such situations and this phenotype was observed occasionally in leaf sections in hp-1 leaves studied. Similar leaf phenotypes to hp-1 are observed when overexpressingphyA in tomato (Boylan and Quail 1989 supra.) and potato (Thiele et al., Plant Physiol. (1999) 102:73-82). The hp-1 leaf phenotype characterized here is consistent with HP-1 acting as a repressive component of a phytochrome-mediated signal transduction pathway and in which hp-1 produces amplified photomorphogenic responses in a highly pleiotropic phenotype (Peters et al., 1998 supra.).

The observation of increased plastid number as well as plastid size in hp-1 cells is novel and demonstrates the possibility of increasing the extent of the plastid compartment size in cells. In particular, fruit pericarp cells have a relatively low proportion of plastid cover, even in hp-1, and there is considerable potential for increasing it greatly. The data suggest that the bulk of plastid division during tomato fruit development occurs during the green stages prior to the breaker stage when the fruit is in transition from green to red. Thus the majority of plastid division occurs as chloroplast division and not chromoplast division.

However, the mechanism by which hp-1 increases plastid number and size is less clear. There have been few studies linking photomorphogenic signal transduction to the cellular control of plastid compartment size. A possible intermediary in any such signalling system could be cytokinin. Since the addition of exogenous cytokinins to wild type Arabidopsis can phenocopy the photomorphogenic det mutant phenotype in the dark (Chory et al. Plant Physiol. (1994) 104:339-347) and cytokinins promote many photomorphogenic responses, including the development of chloroplasts (Huff and Ross, Plant Physiol (1975) 56:429433) and chloroplast division in moss (Kasten et al. 1997), cytokinin could be the link between photomorphogenic signal transduction and plastid division and expansion. This supported by recent evidence of a hypersensitivity of the hp-1 mutant to exogenous cytokinin in the light (Mustilli et al. Plant Cell (1999) 11:145-157). Amongst the downstream targets of any such light-induced plastid division would be the FtsZ genes, which function in the constriction mechanism of dividing plastids (Osteryoung et al. Plant Cell (1998) Current Opinion in Plant Biology 1:475-479). However, although simple perturbation of FtsZ gene function can result in few enlarged plastids in leaf cells (Osteryoung et al. 1998 supra.) the total plastid compartment size is maintained. Since both plastid number density and plastid size is increased in hp-1 cells, it is likely that signals which regulate these two processes may have overlapping components. The change in endpoint of plastid division and plastid expansion control in hp-1 cells suggests that with a greater understanding of the signalling pathways which control plastid division and expansion and their co-ordination, manipulation of plastid compartment size in cells should be feasible.

Suitably, the transformation is effected such that the plants are stably transformed, such that the trait is maintained in the progeny.

Plants obtained using the method of the invention, and progeny and seeds thereof form a further aspect of the invention.

Examples of suitable tissue-specific promoters include the promoters of tomato fruit-specific E8, polygalacturonase invertase, ACC oxidase, fibrillin, rice endosperm, glutelin, wheat endosperm, and RUBISCO cab genes.

Examples of tomato fruit promoters are the E8, tomato polyglacturonase, tomato invertase, Lycopersicon pimpinellifollium invertase, tomato ACC oxidase, and pepper fibrillin promoter. Examples of cereal grain promoters are the rice glutellin promoter, wheat HMW glutenin promoter, maize ESR (Bonello et al. 2000) and Betl 1 promoters (Hueros et al., 1999), the END1 promoter from barley (Doan et al., 1996) the wheat high molecular glutenin (REF required), rice PCNA (expressed in proliferating cells i.e. endosperm and meristems) and blz2 (transcription factor). Examples of cereal leaf promoters are the maize MS8-15, maize chlorophyll a/b binding protein (Cab) (Sullivan et al., 1989), rice RTBV promoter and the maize phosphoenolpyruvate promoter.

Other crop tissue-specific promoters can be obtained by conventional methods. For example, differential screening of DNA libraries from the desired crop tissue as compared to other plant tissue can be used to identify genes that are expressed only in the crop tissue. Such methods are illustrated with respect to the isolation of germination specific promoters in for example International Patent Publication No.97/35983.

In outline, a range of partial clones are amplified by reverse transcriptase polymerase chain reaction (RT-PCR) on RNA from the target crop tissue. Preliminary assessment of the clones' expression in the crop tissue can be made by northern blotting. More detailed northern blot experiments on selected clones may be carried out, for example assess their time course of expression. A cDNA library is then constructed from tissues showing a high expression of these clones, followed by screening of a genomic library to subclone the promoter areas. Final assessment of the spatial and temporal regulation of the cloned promoters may be conducted by transcriptional fusion of the promoter fragments with a reporter gene such as the β-glucuronidase (GUS) reporter gene and transformation into a test plant such as tobacco.

In one embodiment of the invention, the tissue-specific promoter controls expression of phytochrome A. By introducing at least one further copy of the coding sequence into the plant in this way, it will ensure that phytochrome A is overexpressed in the crop part such as the fruits of the plants.

Suitably the phytochrome A gene employed is the native phytochrome A sequence of the plant being transformed. However, this is not necessarily the case as heterologous genes which encode proteins with similar function from different species (and may have some degree of structural homology and so be homologous), a synthetic gene, a chimeric gene or shuffled gene incorporating similar activity may be used. It may be preferable to use a heterologous gene, especially where the gene, or RNA or protein is regulated differently to that of the homologous gene. For example, monocotyledenous genes may be suitably employed in dicotyledenous plants and vice versa. In addition, use of a heterologous gene may reduce problems associated with endo-regulation and/or gene silencing.

A particular example of a phytochrome A gene for use in the method of the invention is phytochrome A from a monocotyledenous plant such as oat phytochrome A as described by Boylan et al. (1989) (supra.), or homologues thereof.

In an alternative embodiment, the construct used to transform the plant cell comprises a sequence which encodes a moiety capable of downregulating a negative regulator of phytochrome A signal transduction. Suitably the negative regulator of phytochrome A signal transduction is the SPA1 or the HP1 gene.

The SPA1 gene and its function as a negative regulator specific for phytochrome A activity has been identified in Arabidopsis (Hoecker et al., 1998, Plant Cell, 10:19-33). Homologous genes which encode proteins with similar function may therefore be identified in other plant species using conventional methods. In particular, sequences based upon the known Arabidopsis sequence (Hoecker et al., Science (1999) 284 496-499) may be used as probes to identify similar sequences in DNA libraries of other plant species.

Once identified in this way, suitable constructs which would result in the downregulation of the gene can be produced.

Downregulation may be achieved in various ways, either at the DNA level, for example using sense, antisense or partial sense technology, or recombinase enzymes, or at the protein level, for example by expression of inhibitor peptides or proteins.

Antisense technology will comprise the generation of “antisense” RNA and “partial-sense” technology requires the generation of “sense” RNA encoding at least part of the functional gene product.

“Antisense RNA” is an RNA sequence which is complementary to a sequence of bases in the corresponding mRNA: complementary in the sense that each base (or the majority of bases) in the antisense sequence (read in the 3′ to 5′ sense) is capable of pairing with the corresponding base (G with C, A with U) in the mRNA sequence read in the 5′ to 3′ sense. Such antisense RNA may be produced in the cell by transformation with an appropriate DNA construct arranged to generate a transcript with at least part of its sequence complementary to at least part of the coding strand of the relevant gene (or of a DNA sequence showing substantial homology therewith).

“Sense RNA” is an RNA sequence which is substantially homologous to at least part of the corresponding mRNA sequence. Such sense RNA, or partial sense RNA, may be produced in the cell by transformation with an appropriate DNA construct arranged in the normal orientation so as to generate a transcript with a sequence identical to at least part of the coding strand of the relevant gene (or of a DNA sequence showing substantial homology therewith). Suitable sense, or partial sense constructs may be used to inhibit gene expression (as described in International Patent Publication WO91/08299).

At the DNA level, either sense or anti-sense oligonucleotides, complementary to the mRNA which corresponds to the target gene (i.e. the gene which encodes the negative regulator of phytochrome A signal transduction) may be generated. When these oligonucleotides are produced in the crop parts of the transformed plant, under the control of the crop tissue specific promoter, they will bind to any mRNA corresponding to the negative regulator of phytochrome A signal transduction, thus preventing translation of the mRMA to protein.

Alternatively, recombinase enzymes may be employed to delete at least some and preferably all of the gene encoding the negative regulator of phytochrome A signal transduction. In this case however, it may be necessary also to engineer recognition sites for the recombinase enzyme into the genome of the plant such that these sites flank the target gene. Then the recombinase enzyme is placed under the control of the crop tissue specific promoter in the method of the invention. When the crop-specific promoter becomes active, recombinase enzyme is produced which interacts with the recognition sites present in the genome of the plant to excise the material therebetween. This will include at least some and preferably all of the gene encoding the negative regulator of phytochrome A signal transduction, which is thereby inactivated in the crop tissue.

Suitable recombinase systems are well known in the art, and include the Cre-Lox system (U.S. Pat. No. 4,959,317), the FLP system (Lyznik et al., Nucleic Acids Research, 1993, Vol. 21, 4, 969-975), the SR1 system (Onouchi et al., Nucleic Acids Research, 1991, 19, 23, 6373-6378) and Gin systems (Maeser et al., Mol. Gen. Genet., 1991, 230:170-176).

In yet a further embodiment, downregulation may be effected by expression of a peptide or protein which inhibits the negative regulator of phytochrome A signal transduction itself, for example, by binding to the protein such that it is no longer able to interact with the phytochrome A pathway.

Such peptides may be identified using routine assay methods. Once the gene encoding the negative regulator of phytochrome A signal transduction has been identified, the protein may be produced or isolated. Peptides which bind to said protein may then be identified using conventional binding assays or, genes encoding them by the yeast 2-hybrid system. Tests can be used to determine whether the protein/peptide complex is still active in the phytochrome A pathway. In general, these methods would be effected in vivo. For example, the complex may be microinjected into a plant cell and the effects determined using reporter genes if possible. Alternatively, the cells can be transformed with a sequence which encodes the peptide and the activity/phenotype of the transformed cells determined.

Alternatively, the peptides may comprise small fragments of the negative regulator of phytochrome A signal transduction. Suitable fragments are those which bind to receptor proteins in the pathway, thereby preventing the binding of the negative regulator of phytochrome A signal transduction itself. The fragments are however inactive, and thus block the activity of the negative regulator.

In a further alternative embodiment, the construct used to transform the plant cell comprises a sequence which encodes a moiety capable of upregulating a positive regulator of phytochrome A signal transduction, in particular where the regulator constitutes a rate-limiting step in the phytochrome A pathway. Examples of positive regulators include PHY1 and PHY3.

Upregulation may be achieved by transforming the plant with genes encoding the regulator or proteins which have similar function, for example heterologous genes, chimeric or synthetic genes or using any other strategy, as outlined above in relation to the upregulation of phytochrome A itself.

In a further embodiment of the invention, the construct used to transform the plant cell comprises a sequence which encodes a moiety capable of downregulating the HP-1 gene. Downregulation of this gene may be effected at the DNA, RNA or protein level, using conventional methods such as those outlined above in relation to the downregulation of negative regulators.

As used herein, the term “homologues” or “homologous genes” includes variants including allelic variants, and genes which have some degree of similarity of structure to the gene in question, and encode proteins with a similar function, but which are derived from other plant species. Suitably the level of structural similarity between the basic gene and the homologue is at least 50%, for example, at least 70%, preferably at least 80%.

Generally speaking, homologues or homologous genes may be identified either by comparison of sequence data using any of the known homology programmes, for example, those exemplified below, or by conducting hybridisation experiments.

The hybridisation experiments will identify genes or DNA which hybridise to the basic DNA. Preferably, such hybridisation occurs at, or between, low and high stringency conditions. In general terms, low stringency conditions can be defined as 3×SCC at about ambient temperature to about 65° C., and high stringency conditions as 0.1×SSC at about 65° C. SSC is the name of a buffer of 0.15M NaCl, 0.015M trisodium citrate. 3×SSC is three times as strong as SSC and so on.

The term “variant” includes DNA sequences which encode sequences of amino acids which differ from the base sequence from which they are derived in that one or more amino acids within the sequence are substituted for other amino acids, but which retain similar biological activity. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will have levels of similarity of at least 60%, preferably at least 75%, and more preferably at least 90% to the base sequence.

As used herein the term “level of similarity” is used to denote sequences which when aligned have similar (identical or conservatively replaced) amino acids in like positions or regions, where identical or conservatively replaced amino acids are those which do not alter the activity or function of the protein as compared to the starting protein. For example, two amino acid sequences with at least 85% similarity to each other have at least 85% similar (identical or conservatively replaced amino residues) in a like position when aligned optimally allowing for up to 3 gaps, with the proviso that in respect of the gaps a total of not more than 15 amino acid resides is affected. The degree of similarity may be determined using methods well known in the art (see, for example, Wilbur, W. J. and Lipman, D. J. “Rapid Similarity Searches of Nucleic Acid and Protein Data Banks.” Proceedings of the National Academy of Sciences USA 80, 726-730 (1983) and Myers E. and Miller W. “Optimal Alignments in Linear Space”. Comput. Appl. Biosci. 4:11-17(1988)). One programme which may be used in determining the degree of similarity is the MegAlign Lipman-Pearson one pair method (using default parameters) which can be obtained from DNAstar Inc, 1228, Selfpark Street, Madison, Wis., 53715, USA as part of the Lasergene system.

Particular plants which may be transformed using the method of the invention include fruit-producing plants such as tomatoes and bananas, grains such as rice, wheat and maize, leaf based vegetables such as lettuce, cabbage and spinach, and root vegetables such as carrot and potato. The tissue-specific promoter employed will be selected to suit the plant being transformed, as outlined above.

Transformation of the plant cells may be effected using any of the conventional methods. In particular, methods such as Agrobacterium transformation, bombardment or via whiskers may be used, depending upon the particular plant species being transformed.

In a further aspect the invention comprises DNA constructs for use in the method of the invention. Thus, in one embodiment, the invention provides a DNA construct comprising a tissue-specific promoter which is specific for crop tissue of the plant, and a sequence encoding phytochrome A, under the control of said tissue-specific promoter.

In another embodiment, the invention provides a DNA construct comprising a tissue-specific promoter which is specific for crop tissue of the plant, and a sequence encoding a moiety which downregulates a negative regulator of phytochrome A signal transduction, or upregulates a positive regulator of phytochrome A signal transduction, under the control of said tissue-specific promoter. Suitable moieties are those described above in relation to the method of the invention.

A further aspect of the invention comprises a plant transformation vector which includes a DNA construct as defined above.

The invention will now be particularly described by way of example with reference to the accompanying diagrammatic drawings in which

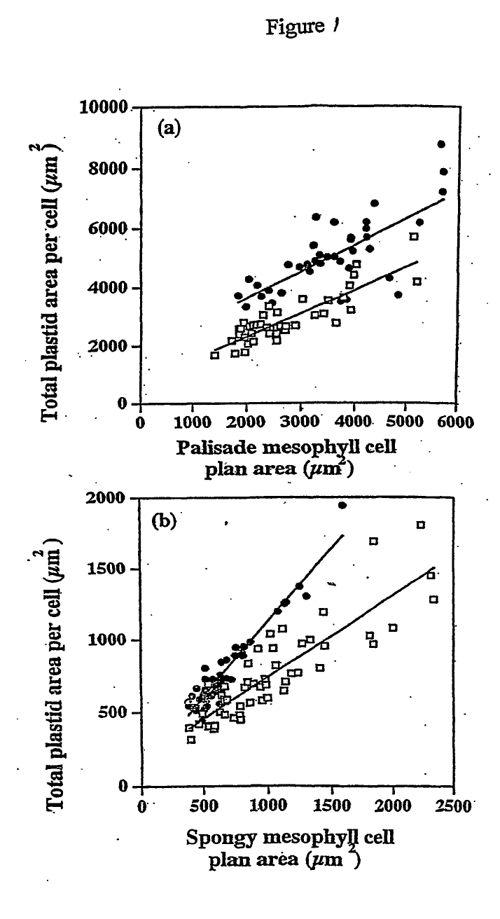

FIG. 1 illustrates the relationship between total chloroplast plan area per cell and (a) palisade and (b) spongy mesophyll cell plan area for wild type tomato cv Ailsa Craig (□) and the hp-1 mutant(λ);

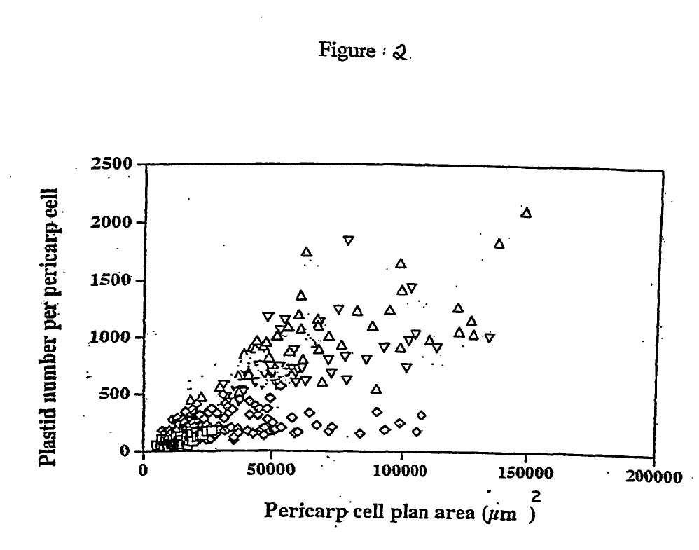

FIG. 2 shows the relationship between plastid number per cell and pericarp cell plan area during the development of the tomato fruit in cv Ailsa Craig. The developmental stages are IG(□), MG (⋄), breaker (∘), 4-days post-breaker (Δ) and 9 days post-breaker (mature ripe) (∇);

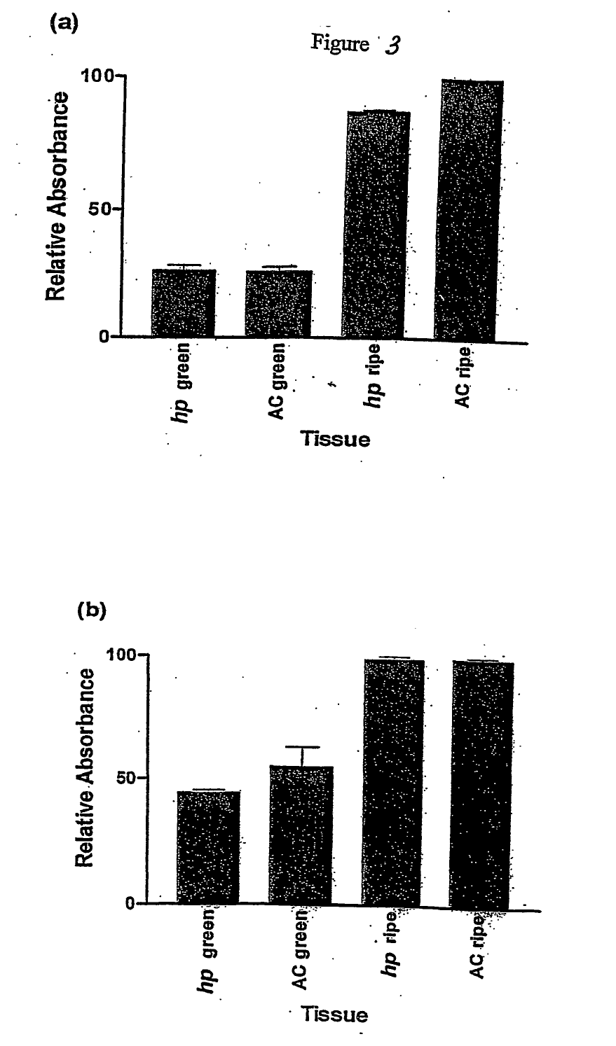

FIG. 3 shows the expression of phytoene desaturase and phytoene synthase-1 in hp-1 and Ailsa Craig fruit where (a) shows the relative expression of Psy-1 in hp and Ailsa Craig (n=3). Psy-1 expression in the hp-1 mature green fruit relative to Ailsa Craig (AC) (where AC=1.00) is 1.01±0.01. Psy-1 expression in hp ripe fruit relative to AC (where AC=1.00) is 0.869±0.007; and (b) shows relative expression of Pds in hp and Ailsa Craig (n=3). Pds expression in hp mature green fruit relative to AC (where AC=1.00) is 0.823+0.1. Pds expression in hp ripe fruit relative to AC (where AC=1.00) is 0.994+0.006;

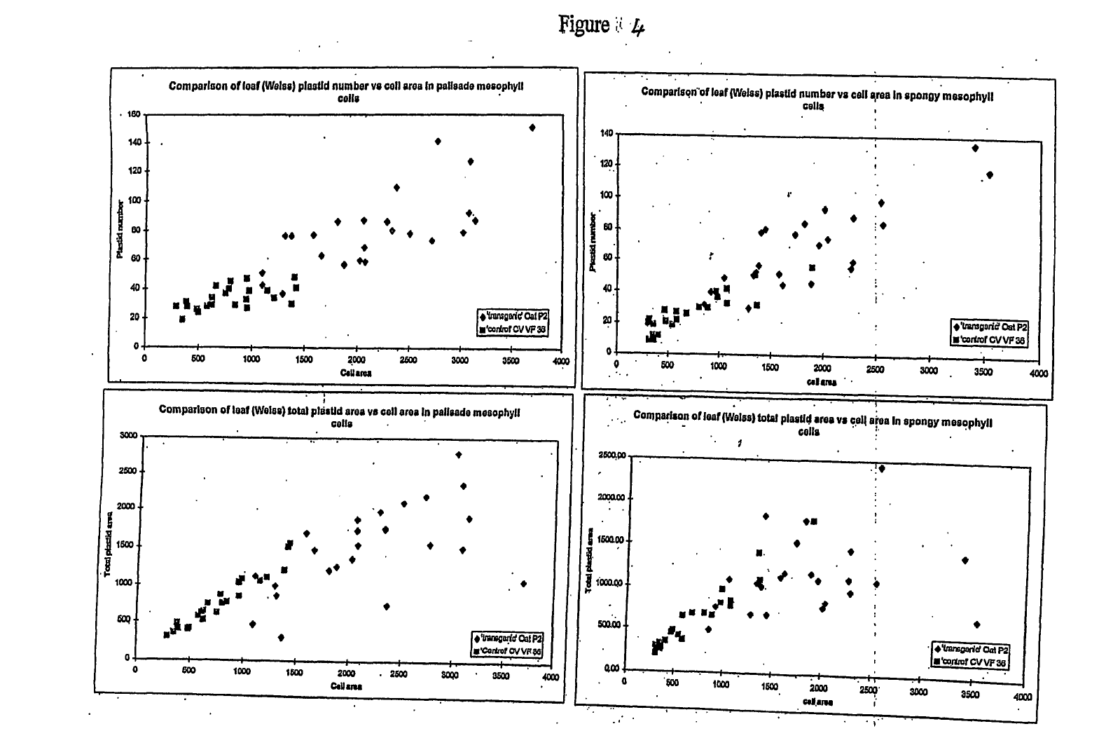

FIG. 4 illustrates a comparison of the phenotype of a transgenic tomato which expresses oat phytochrome A (□) and a control ( [symbol] ) cv VF 36—where (a) shows a comparison of leaf plastid number vs cell area in palisade mesophyll cells, (b) shows a comparison of leaf plastid number vs cell areas in spongy mesophyll cells, (c) shows a comparison of leaf total plastid area vs cell area in palisade mesophyll cells and (d) shows a comparison of leaf total plastid area vs cell area in spongy mesophyll cells.

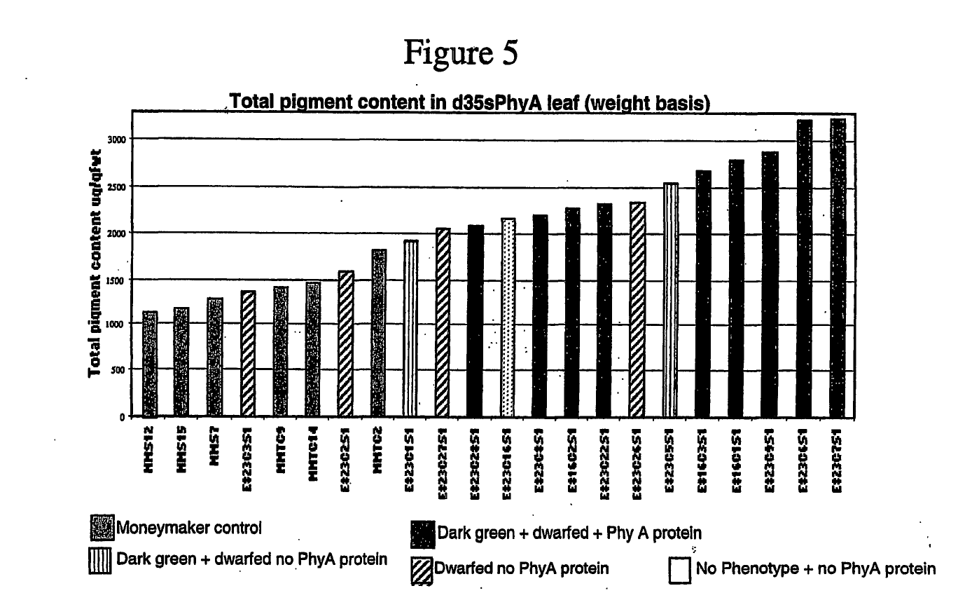

FIG. 5 shows the analysis of the chlorophyll A and B and carotenoid content, on a weight basis) of leaves of transgenic plants expressing oat phytochrome A under control of the double CaMV35S promoter.

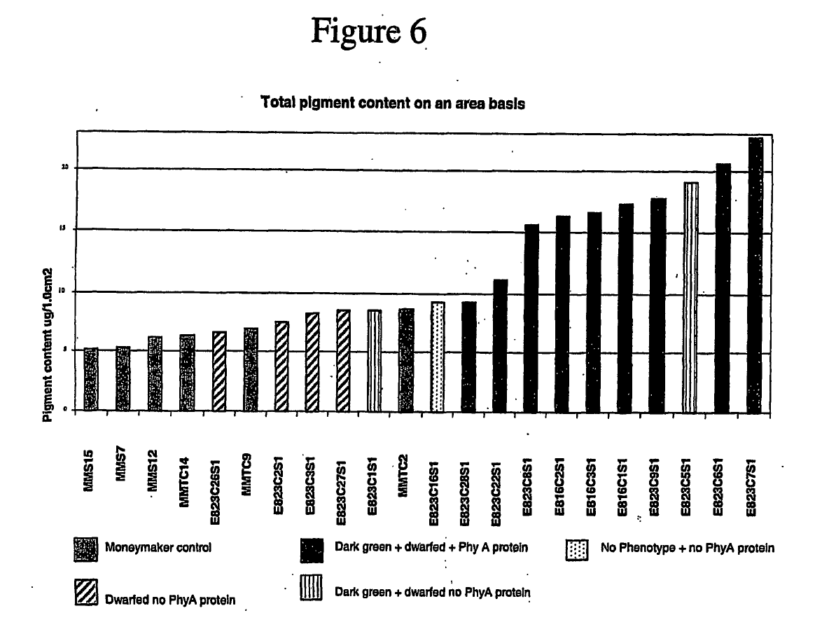

FIG. 6 presents essentially the same data as FIG. 5 but with concentrations recalculated as pigment content on an area basis.

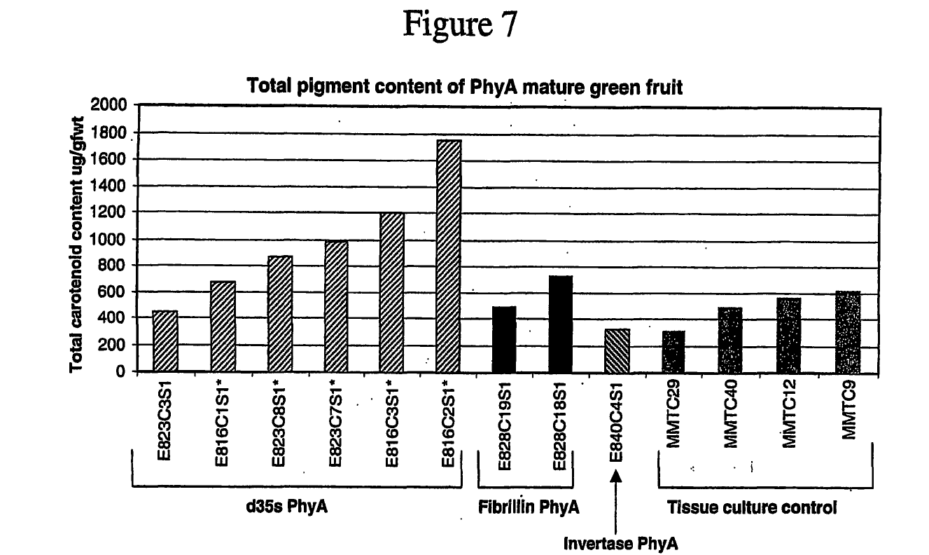

FIG. 7 shows the total pigment content of mature green tomato fruit expressing PhyA.

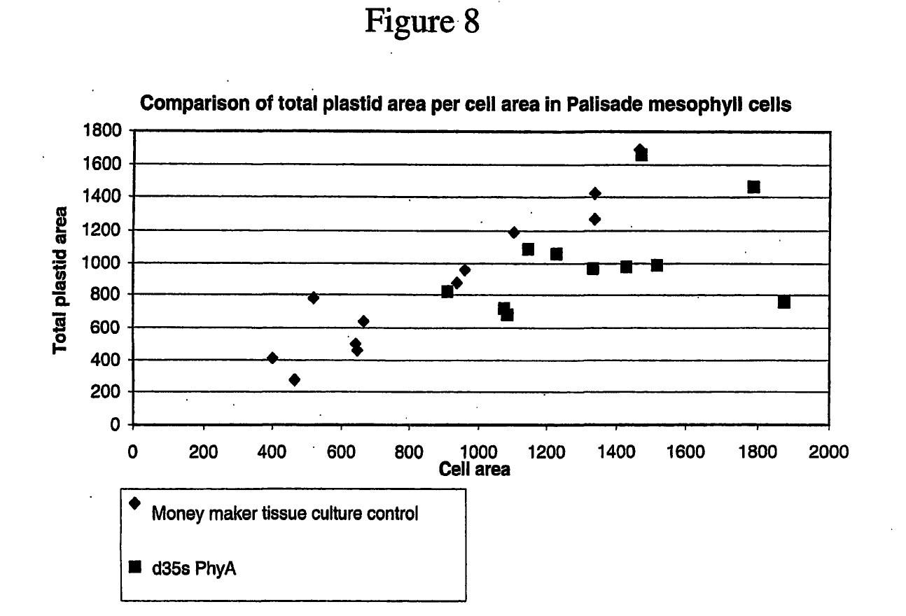

FIG. 8 shows a comparison of the total plastid area per cell area in palisade mesophyll cells.

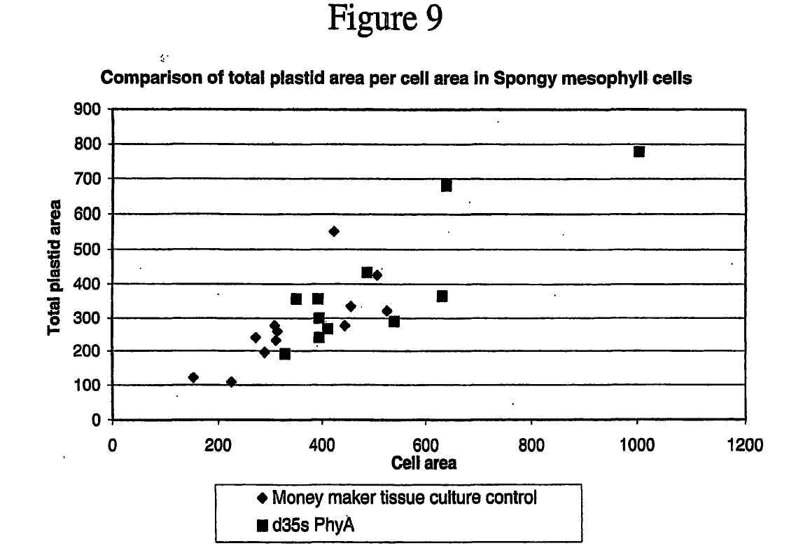

FIG. 9 shows a comparison of the total plastid area per cell area in spongy mesophyll cells.

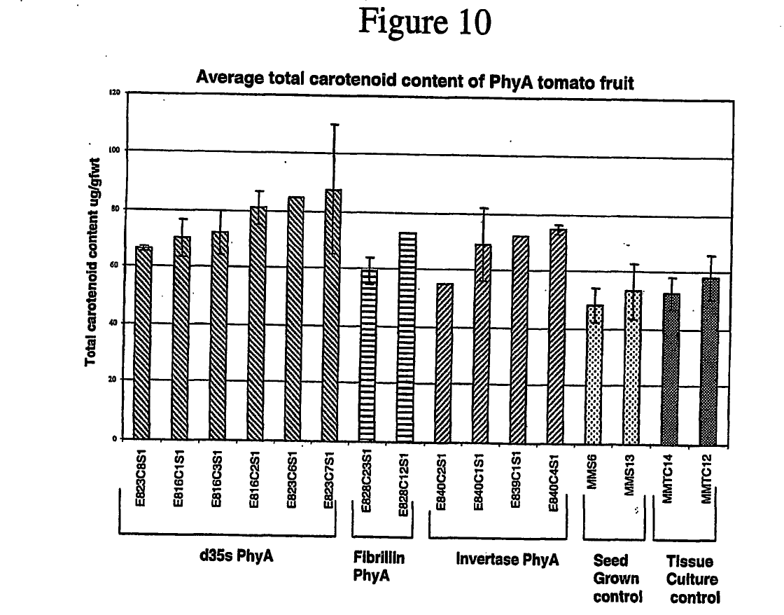

FIG. 10 shows the average total carotenoid content of tomato fruit expressing PhyA.

The invention will now be described by way of illustration in the following Examples. The polynucleotides used as components of the constructs used for transformation of tomato in the Examples below are the double CaMV35S promoter, the fibrillin promoter, the yomato invertase promoter and the coding region of the phytochrome A gene of oat. The termination region, including the polyadenylation signal, was the widely used 3′ untranslatecd region of the nopaline synthase gene of Agrobacterium tumefaciens. The sequences of these components may be found in the following references, incorporated herein by reference. Double CaMV35S (d35S) promoter: U.S. Pat. Nos. 5,164,316 and 5,322,938: GENSEQN Accession Nos. AAQ31609, AAQ81755 and AAQ81756. See also Kay et al. Science (1987) vol. 236, pages 1299-1302

Fibrillin promoter: International Patent Application No. WO 99/16879, GENESEQN Accession No. AAX34367.

Tomato invertase promoter: Elliot et. al., Plant Molecular Biology, 21:515-524 (1993); EMBL Accession Nos. Z12025, Z12026, Z12027 and Z12028.

Phytochrome A gene: Boylan & Quail, The Plant Cell, (1989) 765-777; Hershey et. al. Nucleic Acids Research, 135, 8543-8559 (1985).

### EXAMPLE 1

Study of hp-1 Mutant of Tomato Seeds of tomato (Lycopersicon esculentum cv Ailsa Craig) and an isogenic line homozygous for the hp-1 mutation were sown in a 4:1 vermiculite/granular-clay-based compost mixture and transferred to individual 22 cm diameter pots when the first true leaves appeared. Mutant and wild type plants were arranged on a greenhouse bench in a randomised block design. Tissue samples from the fourth leaf were harvested at noon, 21 days after sowing (defined as immature tissue) whereas mature fully expanded leaf tissue was harvested 56 days after sowing. Tomato fruit were harvested at different ages after anthesis and were classified as immature green (IG, 20 DPA) and mature green (MG, 35 DPA). Ripening fruit were related to the breaker state (40 DPA) when the fruit starts to turn from green to orange. Ripening fruit were harvested at four and nine days post breaker, the latter stage representing fully mature ripe fruit.

Leaf Tissue Fixation and Analysis

Pieces of leaf tissue 1 mm² were cut from midway along immature and fully expanded fourth leaves with a razor blade and fixed immediately in 3 (v/v) gluteraldehyde, 4% (v/v) formaldehyde in 0.1 M PIPES buffer (pH 7.2) for 1 h. Further fixation and subsequent embedding in Spurr's resin was carried out as previously described for Arabidopsis leaf tissue (Kinsman and Pyke, 1998, 125: 1815-1822). For both mutant and wild type, at least six different fourth leaves were sampled. For analysis of internal leaf anatomy, transverse sections 0.5 μm thick were cut using a Cambridge Huxley II microtome, mounted on glass slides and stained with 1% (w/v) toluidine blue for 5 seconds at 60° C. Leaf thickness and parameters of internal leaf morphology were measured using Lucia image analysis software and a Nikon Optiphot microscope (Kinsman and Pyke, Development, 1998, supra.).

Determination of Plastid Compartment Size in Intact Cells

Plastid compartment size was determined by counting plastid number and measuring plastid size in intact fixed separated cells, using previously described methods for Arabidopsis leaves (Pyke and Leech, Plant Physiol. (1991) 96: 1193-1195). Both leaf and tomato fruit pericarp tissue were harvested and fixed immediately in 3.5% (v/v) gluteraldehyde solution for 1 h in the dark. Leaf tissue was subsequently processed as described previously (Pyke and Leech 1991 supra.). Pericarp cells from tomato fruit are more easily separated than leaf mesophyll cells and require less harsh treatment. Therefore pericarp tissue from green tomato fruit was heated in 0.1 M Na₂-EDTA at 60° C. for 10-30 min in an effort to minimise cell damage whereas orange and red fruit pericarp tissue was stored in Na₂-EDTA solution at 4° C. Both of these procedures allowed the subsequent separation of intact pericarp cells on a microscope slide after gentle tapping of the tissue with forceps. Numbers of chloroplasts and chromoplasts in leaf mesophyll cells and fruit pericarp cells were determined in intact, separated cells using Nomarski differential interference contrast optics on a Nikon Optiphot microscope. Plan areas of individual cells and plastids within them were measured by image analysis (Kinsman and Pyke, 1998 supra.). Total plastid area was expressed as a product of mean plastid area per cell×plastid number per cell. A cell index parameter was calculated as a measure of the total plastid compartment size in relation to cell size and was calculated as total plastid are per celucell plan area.

Pigment Analyses

Carotenoids and chlorophylls were extracted from tissues and then analysed spectrophotometrically and by HPLC, as described previously (Fraser et al., Plant Physiol (1994) 105: 405-413).

Reverse Transcriptase-PCR

Total fruit RNA was extracted from fresh frozen tomato fruit pericarp tissue using the method described by Schultz et al. (Plant Molecular Biology Reporter (1994), 12: 310-316) with the following modifications. After incubation in the homogenising buffer, the aqueous phase was extracted twice in an equal volume of phenol: chloroform and then again in chloroform. Nucleic acids were recovered by ethanol precipitation. The RNA was precipitated by adding 12M LiC 1 to a final concentration of 2M and incubation overnight at 4° C. It was pelleted by centrifugation at 10,000 g, rinsed in 70% ethanol and then dissolved in dH₂O.

RNA was denatured by incubating each sample (1 μg) and the external control (25 ng), at 65° C. for 5 min. The samples were chilled on ice and added to a mixture containing 3′end oligo(dT)₁₅ primer (100 ng); 1U Rnasin ribonuclease inhibitor; 1 mM dNTPs; 1× reaction buffer (250 mM Tris-HCl, pH 8.3, 375 mM KCl, 15 mM MgCl₂, 50 mM dithiothreitol) and 5U of Moloney murine leukaemia virus reverse transcriptase (M-MLV RT). Reverse transcription of the total RNA samples was performed for 1 hour at 37° C., and the sample then heated at 98° C. for 5 min to terminate the reaction.

Oligonucleotides used for amplification of phytoene synthase-1 (Psy-1) were: upstream, 5′CAGCCTTAGATAGGTGGG 3′ from 724-741 bp and downstream, 5′GAGTCTACTTGCCTCAAG 3′ from 1082-1104 bp, giving rise to a 380 bp PCR product. Oligonucleotides used for amplification of phytoene desaturase (Pds) were: upstream, 5′CTGAATGAGGATGGAAGTGTCAAG 3′ from 1459-1482 bp and downstream, 5′CTACACGAAACAGAAATACTTGGC 3′ from 2013-2027 bp giving rise to a 568 bp PCR product.

The external control was rat transthyretin-specific primers. The external control RNA used in the procedure originated from rat hepatoma cells. 2.5 ng of total RNA was used with primers specific for transthyretin mRNA (Pecker et al., 1996) with the following primers: upstream 5′AGTCCTGGATGCTGTCCGAG 3′ from 139-158 bp, and downstream 5′CTTGGCATTTCCCCATTCCATG 3′ from 338-359 bp, giving rise to a 180 bp PCR product.

PCR was performed at a final concentration of 1×PCR buffer (20 mM Tris-HCL, pH8.0, 500 mM KCl); 2.5 mM MgCl₂; 1 mM dNTPs; 10 ng each primer; and 2.5U Taq DNA polymerase (Gibco BRL). The reaction was heated to 95° C. for 5 min. Amplification was performed in 30 sequential cycles of 94° C., 1 min; 50° C., 30s; and 72° C., 30s. After the last cycle the samples were incubated for an additional 10 min at 72° C. PCR products were separated by electrophoresis on 1% (w/v) agarose gels containing 1×TBE and 0.1 μg/mL ethidium bromide. Band intensities were quantified by scanning the gels using the UVP Gel Documentation and Analysis System, GDS 7600 pre-loaded with Gel Base/Gel Blot Pro Software. Sample-to-sample variation in RT and PCR efficiency was corrected for by using the relative quantity of the control PCR product. All PCR products were sequenced and identified as the targeted mRNA by alignment with their corresponding sequences in the database.

Phytoene Synthase Enzyme Assay

Phytoene synthase activity was assayed in plastid preparations using the protocol of Fraser et al. (1994) supra.

Other Determinations

Measurements of ploidy levels were carried out by Plant Cytometry Services, Schijndel, The Netherlands. Protein levels were determined by the method of Lowry et al. (1951), following precipitation (Wessel and Flugge, Anal. Biochem, (1984) 138:141-143).

Results

Carotenoid and Chlorophyll Content of Leaf and Fruit

Chlorophyll levels in both leaf and mature green fruit were higher in hp-1 than in the wild type (1.20 and 1.38-fold, respectively), but the relative amounts of chlorophyll a and b were virtually the same (Table 1).

TABLE 1

| | Chlorophyll | | | | Chlorophyll |
|---|---|---|---|---|---|
| Variety/ | μg/g | | Chl b | Carotenoid | Carotenoid |
| tissue | FW | Chl a % | distribution | (μg/g FW) | ratio |
| Green Fruit | | | | | |
| Ailsa Craig | 84 | 79 | 21 | 18 | 3.8 |
| hp-1 | 116 | 73 | 27 | 31 | 4.7 |
| Red Fruit | | | | | |
| Ailsa Craig | 0 | 0 | 0 | 165 | na |
| hp-1 | 0 | 0 | 0 | 299 | na |
| Leaf | | | | | |
| Ailsa Craig | 2207 | 66 | 34 | 155 | 14.2 |
| hp-1 | 2648 | 67 | 33 | 171 | 15.5 |

na = not applicable

Similarly, carotenoids were increased in all hp-1 tissues compared with the wild type and especially so in ripe fruit (1.81-fold). A comparison of individual carotenoids in leaf and ripe fruit revealed only very small differences between the two varieties (Table 2).

TABLE 2

| % Distribution | |
|---|---|
| Ripe Fruit | Leaf |

| Carotenoid | Ailsa Craig | hp-1 | Ailsa Craig | hp-1 |
|---|---|---|---|---|
| Phytoene | 9 | 9 | ND | ND |
| Lycopene | 65 | 65 | ND | ND |
| beta-Carotene | 22 | 22 | 22 | 23 |
| Lutein | 5 | 6 | 53 | 50 |
| Neoxanthin | ND | ND | 3 | 2 |
| Violaxanthin | ND | ND | 22 | 25 |

where ND = not detected

Leaf Structural Analysis

In order to determine whether underlying internal leaf morphology contributes to the darker green leaf phenotype observed in the hp-1 mutant, transverse sections taken from the mid-point of the immature and mature fully expanded fourth leaf were analysed. Whereas leaf thickness and internal leaf morphology was similar between hp-1 and wild type in expanding leaves after 21 d, at full expansion the palisade mesophyll cells in hp-1 leaves showed greater periclinal elongation compared to wild type (Table 3).

TABLE 3

| | Ailsa Craig | | hp-1 | |
|---|---|---|---|---|
| Days from sowing | 21 | 56 | 21 | 56 |
| Leaf thickness (n = 6) | 205(15) | 225(2) | 211(18) | 309(3) |
| Palisade cell length | 56.8(0.9) | 77.6(2.3) | 65.1(0.9) | 123(2.6) |
| (n = 150) | | | | |
| Palisade cell width | 14.5(0.2) | 30.0(0.9) | 17.5(0.3) | 33.3(0.5) |
| (n = 150) | | | | |
| Palisade length:leaf | 0.27 | 0.35 | 0.31 | 0.40 |
| thickness | | | | |

Standard errors are shown in parentheses. Dimensions of palisade mesophyll cells were measured in preparations of fixed isolated cells. Leaf thickness was measured from leaf transects.

This results in palisade mesophyll cells contributing to an increased proportion of leaf thickness in the hp-1 mutant leaves. Spongy mesophyll cells were not significantly altered in size or shape in hp-1 leaves (data not shown). Even though mature leaves of the hp-1 mutant showed a significant increase in the periclinal expansion of palisade mesophyll cells, overall leaf shape was unaltered in the mutant, although leaf area was slightly reduced (data not shown).

Leaf Chloroplast Development

In order to determine whether leaf cells in hp-1 also show differences to wild type in chloroplast populations in mesophyll cells, chloroplast numbers and sizes were determined in preparations of fixed isolated mesophyll cells in expanding and fully expanded leaves. In cells of expanding leaves, 21 days after sowing, no difference was observed in plastid number, plastid size or plastid density between wild type and the hp-1 mutant (data not shown). However, in fully expanded leaves of hp-1, chloroplast density, expressed as chloroplasts per unit cell plan area, in both palisade and spongy mesophyll cells was found to be increased compared to wild type (Table 4).

TABLE 4

| | Ailsa Craig | | hp-1 | |
|---|---|---|---|---|
| | Pal | Sp | Pal | Sp |
| Cell plan area (μm², n = 150) | 2708(140) | 1044(70) | 3606(159) | 662(36) |
| Chloroplast number per cell(n = 40) | 119(5) | 43(2) | 157(5) | 36(2) |
| Chloroplast density (per μm² | 0.039 | 0.042 | 0.043 | 0.056 |
| cell plan area) | (0.001) | (0.001) | (0.001) | (0.001) |
| Chloroplast plan area (μm², | 24.2(0.6) | 17.8(46) | 30.0(0.6) | 21.1(0.2) |
| n = 400) | | | | |
| Total plastid area (μm², n = 40) | 2884(132) | 762(46) | 5077(194) | 768(39) |
| Cell index | 1.063 | 0.72 | 1.41 | 1.16 |

Standard errors are shown in parentheses. Cell index is calculated as total plastid area/cell plan area.
Pal = palisade
Sp = spongy mesophyll

In addition, hp-1 chloroplasts in both palisade and spongy cells were larger than wild type. These two characteristics together result in a significantly increased total area of chloroplast per mesophyll cell in the hp-1 mutant and an increased cell index (Table 4). This increased chloroplast compartment size was found across the range of palisade cell sizes observed in fully expanded leaves and also in larger spongy mesophyll cells.

Analysis of Pericarp Cell Plastids in Tomato Fruit

The number of chloroplasts and chromoplasts in individual pericarp cells were counted. The relationship between increasing pericarp cell size and plastid number during fruit development was complex (FIG. 1). In pericarp cells from IG and MG fruit much variation was found in the relationship between plastid number and pericarp cell size and pericarp cells. Extreme differences in plastid number/plastid size combinations were observed (FIG. 2). Between the MG and breaker stages a large increase in plastid number per cell occurred but this did not increase substantially during the subsequent ripening stages of the red fruit. Chloroplast numbers in green fruit ranged between 200-400 per cell, but by the breaker stage plastid numbers had increased dramatically to an average of around 800 per cell, although many larger cells contained up to 2000 chromoplasts per cell. Mature pericarp cells in wild type fruit did not show the same heterogeneity in plastid number/size combinations as shown in green fruit and were typified by being extremely large cells, around 100,000 μm² in plan area with large populations of small red chromoplasts.

Pericarp cells in both MG and fully ripe fruit of hp-1 showed an increased in plastids per cell and plastid size compared to wild type (Table 5), such that both plastid density per cell and cell index increased consistently in hp-1 pericarp cells compared to wild type.

TABLE 5

| | Ailsa Craig | hp-1 | Ailsa Craig | hp-1 |
|---|---|---|---|---|
| | Mature green (30 DPA) | | Ripe (9 DPB) | |
| Cell plan area (μm², n = 150) | 18821(901) | 24904 | 60028 | 56319 |
| | | (1644) | (3472) | (2624) |
| Chloroplast number per | 246(9) | 312(20) | 760(40) | 826(27) |
| cell(n = 40) | | | | |
| Plastid density (per μm² cell | 0.013 | 0.0125 | 0.0127 | 0.0147 |
| plan area n = 50) | | | | |
| Plastid plan area (μm², n > 100) | 12.7(0.3) | 14.8(0.5) | 18.5(0.2) | 20.6(0.3) |
| Total plastid area per cell (μm², | 3102(115) | 4669(332) | 14223(914) | 17186(693) |
| n = 50) | | | | |
| Cell index | 0.165 | 0.187 | 0.237 | 0.305 |

Standard errors are shown in parentheses. Cell index is calculated as total plastid area/cell plan area
Carotenoid Gene Expression and Phytoene Synthase Activity

Expression of either Pds or Psy-1 was not significantly different in mature green and ripe fruit of the two varieties. In contrast, however, the in vitro enzyme activity of phytoene synthase was 1.9 fold higher in hp-1 than Ailsa Craig fruit (data not shown).

Ploidy

Both varieties were found to be diploid.

### EXAMPLE 2

Comparison of Transgenic Tomato Plants expressing Oat Phytochrome A (Promoter CaMV35S) with Wild-Type Variety

The method of Example 1 was repeated using a transgenic tomato variety expressing Oat phytochrome A (obtained from Peter Quail of the University of California) as described in Boylan and Quail, The Plant Cell, (1989) 765-777, and with CV VF 36 variety as the control.

The results are shown in FIG. 4 and Table 2. These results clearly show that the transgenic leaves have a significantly higher cell size.

The results of a statistical analysis of 35sPhyA carotenoid content is shown in Table 6.

TABLE 6

| | | | | | | total |
|---|---|---|---|---|---|---|
| | β-carotene | β-carotene | lycopene | lycopene | total carotenoid | carotenoid |
| Treatment | μg/gfwt | μg/gdwt | μg/gfwt | μg/gdwt | μg/gfwt | μg/gfwt |
| Transgenic | 3.09 | 36.3 | 21.1 | 250 | 24.2 | 286 |
| Control | 1.96 | 24.2 | 17.7 | 215 | 19.6 | 239 |
| F = Test | 0.02% | 0.00% | 2.59% | 1.80% | 0.87% | 0.39% |
| probability | | | | | | |

Carotenoid content is significantly increased in the transgenic variety.

### EXAMPLE 3

Transgenic Tomato Plants Expressing Oat Phytochrome A (dCaMV35S Promoter)

Tomato c.v.Moneymaker was transformed with a construct specifying phytochrome A, as described in Example 2 but using the fibrillin promoter instead of the CaMV35S promoter as described in Example 2.

### EXAMPLE 4

Transgenic Tomato Plants Expressing Oat Phytochrome A (Fibrillin Promoter)

Tomato c.v.Moneymaker was transformed with a construct specifying phytochrome A, as described in Example 2 but using the fibrillin promoter instead of the CaMV35S promoter as described in Example 2.

### EXAMPLE 5

Transgenic Tomato Plants expressing Oat Phytochrome A (Invertase Promoter)

Tomato c.v.Moneymaker was transformed with a construct specifying phytochrome A, as described in Example 2 but using the tomato invertase promoter (Kathryn J Elliot et al (1993) Plant Molecular Biology 21:515-524. isolation and characterisation of fruit vacuolar invertase genes from two tomato species and temporal differences in mRNA levels during fruit ripening) instead of the CaMV35S as described in Example 2.

### EXAMPLES 3, 4 AND 5

### Comparison of Results

Around 30 transgenic plants from each construct described in Examples 3, 4 and 5 were produced. In addition, 20 seed and 20 tissue grown controls were grown. These plants were randomly placed in the glasshouse.

The following analyses were performed on primary To plants
Visual analysis on plant, leaf, mature and ripe fruit phenotype
Leaf carotenoid analysis
Mature green fruit carotenoid analysis
Leaf plastid density measurements
7 dpb fruit carotenoid analysis
Molecular analysis: Western, Northern analysis and copy number analysis
   Plant Visual Analysis

Around 10 d35s PhyA plants showed significant dwarfness and a very dark green leaf phenotype. In the hp mutant and the 35s PhyA plants the seedlings were initially dwarfed but caught up to the control plants height at a later stage. The hp mutant and 35s PhyA plants displayed a dark green leaf phenotype but not as darker green as those in the d35s PhyA leaf material. Here the d35s PhyA plants did not grow out of the dwarfiiess and the dark green leaf colour stayed throughout the age of the plant, this stronger phenotype could be due to the stronger promoter used than in the s35s PhyA plants. There were around 20 d35s PhyA plants that did not show dwarfiiess and dark green leaf phenotype. We have shown in the d35s PhyA plants we have mimicked the hp plant phenotype.

No dwarfism or dark green leaf phenotype were observed in those plants using the fruit specific invertase or fibrillin promoters.

Fruit Visual Analysis

The 10 dark green and dwarfed d35s PhyA plants showed significantly darker colour in the green and ripe fruit (pericarp and columella specifically) as compared to the control. The invertase and fibrillin fruits did not show a significant darker colour than the control fruit.

Leaf Carotenoid Analysis

The pigment content (chlorophyll A+B and carotenoids) of leaf material from d35s PhyA plants were measured on a weight basis and compared to the pigment content in tissue culture and seed grown controls. The results are shows in graphical form in FIG. 5. On a weight basis the transgenics show up to two-fold increase in pigment compared to control. FIG. 6 presents essentially the same data as FIG. 5 but with concentrations recalculated as pigment content on an area basis.

On an area basis the transgenics show up to a 3-fold increase in pigment content as compared to the control.

In the hp mutant on a weight basis there was no significant difference in pigment content as compared to the control, however on an area basis there was up to a 2-fold increase in pigment content as compared to the control. This difference is due to the thickness of the leaf, the hp mutant has a thicker leaf and thus only on an area basis can you detect the difference in pigment content. This data confirms our visual findings in which we noted earlier the d35s PhyA leaves are more pigmented than the hp and control leaf tissue. This result suggests that the transgenics are mimicking the hp phenotype in the d35s phyA constructs.

Mature Green Fruit Carotenoid Analysis

Pigment content (chlorophyll A+B, and carotenoid) from mature green fruit from PhyA transgenics were measured and compared to pigment content in tissue culture grown controls. The results are shown graphically in FIG. 7.

This data shows that the d35s PhyA fruit contain up to a 3-fold more total pigment content than the tissue culture controls. There were no significant differences in total pigment content in the invertase and fibrillin fruit as compared to the tissue culture controls. This data confirms our visual findings seen in the d35s PhyA mature green fruit in which we noted earlier they are more pigmented than the hp and control mature green fruit.

Leaf Plastid Density Measurements

Leaf plastid density was measured in young expanding leaf tissue from d35s PhyA and compared to the tissue cultre control. Total plastid density is the total plastid area in relation to the palisade or spongy mesophyll cells, which holds the plastids. The results are shown graphically in FIGS. 8 and 9.

There were no significant differences in total plastid density in the transgenics compared to the controls.

Fruit Carotenoid Content

Total carotenoid content from 7 dpb fruit from PhyA transgenics were measured and compared to the tissue culture and seed grown controls. The results are shown graphically in FIG. 10.

There is a significant increase in the carotenoid content of fruit from the PhyA transgenics as compared to the controls. This data confirms our visual findings especially for the d35s PhyA fruit as we noted earlier that the pericarp and columella tissue were noticeably darker red in colour than the control and even invertase and fibrillin fruit.

1. A method for increasing the phytonutrient and/or chlorophyll content and/or yield of a crop, said method comprising transforming a plant cell from which viable plants maybe recovered, with a DNA construct comprising a sequence which encodes phytochrome A or a moiety which interacts with the phytochrome A pathway so as to mimic the activity of the hp-1 mutant of tomato, said sequence being under the control of a tissue specific promoter which is specific for a crop tissue of the plant, and thereafter generating viable plants from said cell.

2. A method according to claim 1 comprising transforming a plant cell from which viable plants may be recovered, with a DNA construct comprising either (i) a sequence which encodes phytochrome A, and/or (ii) a sequence which encodes a moiety capable of downregulating a negative regulator of phytochrome A signal transduction, or (iii) a sequence which encodes a moiety capable of upregulating a positive regulator of phytochrome A signal transduction; wherein sequences (i), (ii) and/or (iii) are under the control of a tissue specific promoter which is specific for a crop tissue of the plant, and thereafter generating viable plants from said cell and recovering crop from the plant.

3. A method according to claim 1 comprising transforming a plant cell from which viable plants may be recovered, with a DNA construct comprising a sequence which encodes a moiety capable of downregulating the HP-1 gene, and thereafter generating viable plants from said cell and recovering crop from the plant.

4. The method of claim 1 wherein the phytonutrient is a carotenoid.

5. The method of claim 1 wherein the tissue specific promoter is a fruit-specific promoter.

6. The method according to claim 2 wherein the DNA construct comprises a sequence which encodes a moiety which is able to downregulate a negative regulator of phytochrome A signal transduction.

7. The method according to claim 6 wherein a negative regulator of phytochrome A signal transduction is encoded by a SPA1 gene.

8. The method according to claim 1 wherein the DNA construct comprises a sequence which encodes a phytochrome A.

9. The method according to claim 8 wherein the phytochrome A gene is deregulated.

10. The method according to claim 1 wherein the plant cell is a tomato cell.

11. A transgenic plant obtained by the method claim 1.

12. Seeds or progeny of a plant according to claim 11.

13. A DNA construct comprising a crop-tissue-specific promoter which is specific for crop tissue of the plant, and a sequence encoding phvtochrome A, under the control of said crop-tissue-specific promoter.

14. A DNA construct comprising a tissue-specific promoter which is specific for crop tissue of the plant, and a sequence encoding either a moietv which downregulates a negative regulator of phytochrome A signal transduction, or a moietv which upregulates a positive regulator of phytochrome A signal transduction, under the control of said tissue-specific promoter.

15. A DNA construct comprising a tissuc-specific promoter which is specific for crop tissue of the plant, and a sequence encoding either a moiety which downregulates an HP-1 gene, under the control of said tissue-specific promoter.

16. (cancel)

17. A plant transformation vector compromising the DNA construct of claim 13.

18. A plant transformation vector compromising the DNA construct of claim 15.

19. A plant transformation vector comprising the DNA construct of claim


SEQUENCE LISTING (6 sequences)

SEQ ID NO: 1 - DNA, 18 bp, Artificial
  PCR Primer from Example 1
cagccttagataggtggg

SEQ ID NO: 2 - DNA, 18 bp, Artificial Sequence
  PCR Primer from Example 1
gagtctacttgcctcaag

SEQ ID NO: 3 - DNA, 24 bp, Artificial Sequence
  PCR Primer from Example 1
ctgaatgaggatggaagtgtcaag

SEQ ID NO: 4 - DNA, 24 bp, Artificial Sequence
  PCR Primer from Example 1
ctacacgaaacagaaatacttggc

SEQ ID NO: 5 - DNA, 20 bp, Artificial Sequence
  PCR Primer from Example 1
agtcctggatgctgtccgag

SEQ ID NO: 6 - DNA, 22 bp, Artificial Sequence
  PCR Primer from Example 1
cttggcatttccccattccatg